(19) United States
(12) Patent Application Publication
(10) Pub. No.: US 2005/0131394 A1
(43) Pub. Date: Jun. 16, 2005

(54) Clamping device

(75) Inventors: Todd M. Bjork, River Falls, WI (US); Joseph E. Gleason, Eagan, MN (US)

Correspondence Address: WESTMAN CHAMPLIN & KELLY, P.A., SUITE 1600 - INTERNATIONAL CENTRE, 900 SECOND AVENUE SOUTH, MINNEAPOLIS, MN, 55402-3319, US

(73) Assignee: Minnesota Scientific, Inc., St. Paul, MN (US)

(21) Appl. No.: 10/732,742

(22) Filed: Dec. 10, 2003

(51) Int. Cl. A61B 17/00

(52) U.S. Cl. 606/1

(57) ABSTRACT

A device for positioning a surgical apparatus in a selected position within a surgical site includes a housing attached to a rod positioned over the surgical site. The housing includes an internal cavity into which a force providing mechanism is positioned. A clamping mechanism is attached to the housing wherein the clamping mechanism includes a bore for engaging the surgical apparatus. The force providing mechanism moves to secure the surgical apparatus to the clamping mechanism in the selected position within the surgical site.

### CROSS REFERENCE TO RELATED APPLICATION(S)

None.

### BACKGROUND OF THE INVENTION

The present invention relates generally to a surgical clamp for use in mounting a surgical apparatus within a surgical site. More particularly, the present invention relates to a clamping device that precisely repositions a surgical apparatus in a selected position within a surgical site.

Many surgical procedures that are performed minimize access to a surgical site to minimize trauma to a patient. An exemplary procedure where access to the surgical site is minimized is a spinal procedure performed within a spinal cavity.

After the incision is made, a hole is bored through a vertebrae making up a portion of the spinal column. A surgical apparatus is typically positioned in a selected position within the hole to perform the initial steps of the surgical procedure.

Once initial steps of the surgical procedure are performed, the surgical apparatus is removed from the surgical site to provide access for additional surgical equipment. The additional surgical equipment is used to perform additional steps in the procedure. After the additional steps have been performed, the original surgical apparatus is precisely repositioned in the selected position within the surgical site to prevent additional trauma from occurring to the spinal cavity.

The surgical apparatus is typically clamped in the selected position to a support rod that extends over the surgical site. The support rod is clamped to a surgical support apparatus that is positioned about the surgical site. The surgical apparatus is removed from the surgical site by either removing the surgical apparatus from the support rod or removing the support rod from the surgical support apparatus.

By removing the surgical apparatus from the support rod or moving the surgical rod with respect to the surgical support apparatus, precisely repositioning the surgical apparatus in the selected position within the surgical site is difficult. Therefore, the patient may incur additional unnecessary trauma from the imprecise repositioning of the surgical apparatus within the surgical site.

### SUMMARY OF THE INVENTION

The present invention includes a device for positioning a surgical apparatus in a selected position within a surgical site. The device includes a housing attached to a rod positioned over the surgical site. The housing includes an internal cavity into which a force providing mechanism is positioned. A clamping mechanism is attached to the housing wherein the clamping mechanism includes a bore for engaging the surgical apparatus. The force providing mechanism moves to secure the surgical apparatus to the clamping mechanism in the selected position within the surgical site.

### BRIEF DESCRIPTION OF THE DRAWINGS

FIG. 1 is a perspective view of the clamping device of the present invention proximate a surgical site.

FIG. 2 is a partial exploded view of the clamping device of the present invention.

FIG. 3 is an exploded view of the clamping device of the present invention.

FIG. 4 is a cut away view of the clamping device of the present invention.

FIG. 5 is a sectional view of the clamping member within the housing of the clamping device of the present invention.

FIG. 6 is a perspective view of an alternative embodiment of the clamping device of the present invention.

FIG. 7 is an exploded view of the alternative embodiment of the clamping device of the present invention.

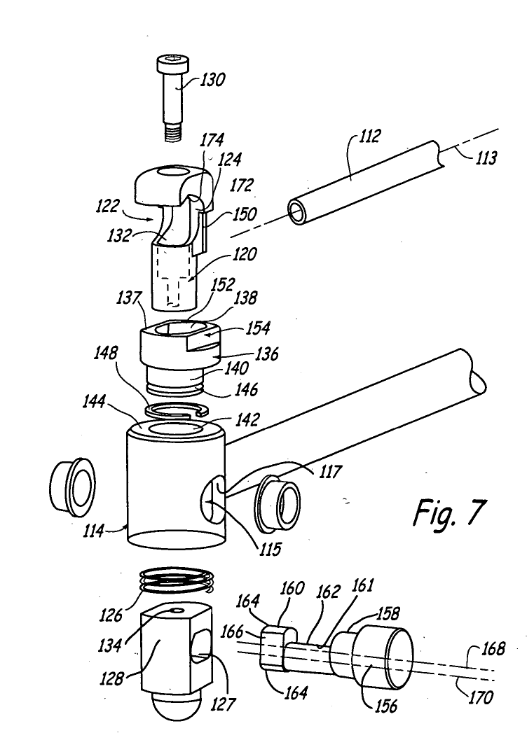

FIG. 8 is a cut-away view of the alternative embodiment of the clamping device of the present invention.

FIG. 9 is a perspective view of another alternative embodiment of the present invention.

FIG. 10 is an exploded view of the alternative embodiment of the present invention.

FIG. 11 is a perspective view of another alternative embodiment of the present invention.

FIG. 12 is an exploded view of the alternative embodiment of the present invention.

### DETAILED DESCRIPTION OF THE PREFERRED EMBODIMENTS

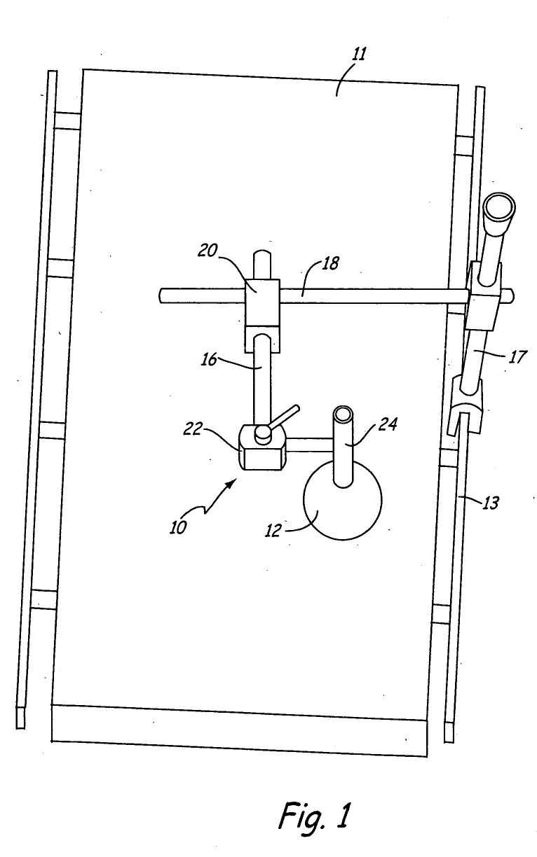

A surgical apparatus-retaining device of the present invention is generally illustrated at 10 in FIG. 1. The surgical apparatus-retaining device 10 secures a surgical apparatus 24 in a selected location within a surgical site 12. A housing 22 of the surgical apparatus-retaining device 10 is fixedly attached to an end of a support rod 16 that extends over the surgical site 12 where the support rod 16 is attached to a support apparatus 18 with a clamp 20. The support apparatus 18 is attached to a field post 17 that is attached to a rail 13 of a surgical table 11. The surgical apparatus-retaining device 10 allows the surgical apparatus 24 to be removed from the surgical site 12 and precisely repositioned in the selected location within the surgical site 12 provided the surgical apparatus-retaining device 10 remains stationary during a surgical procedure.

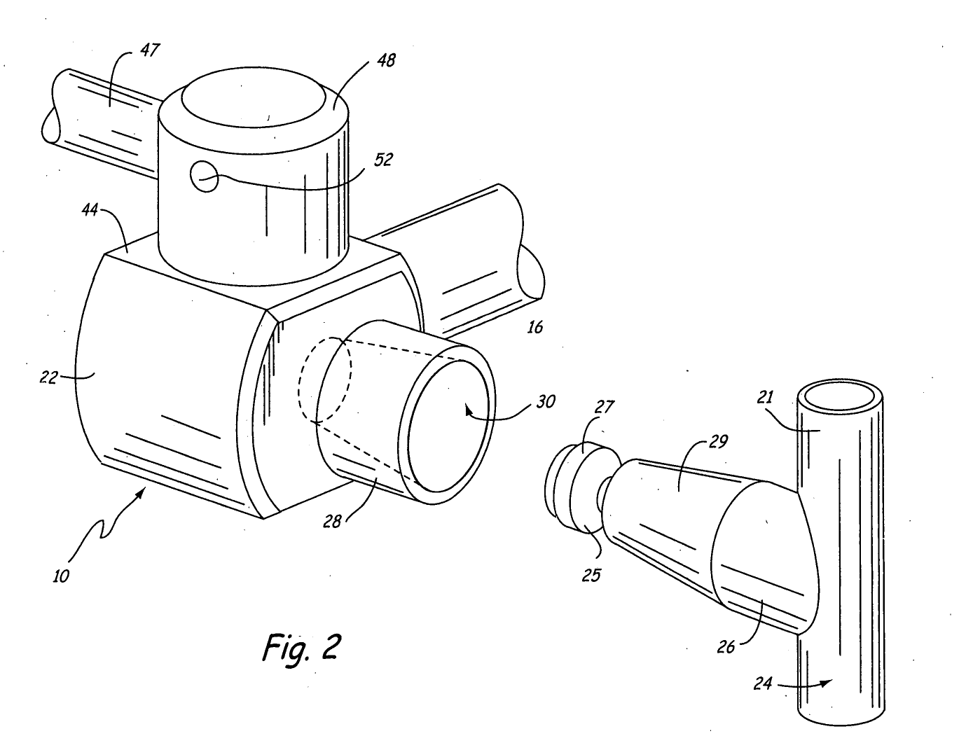

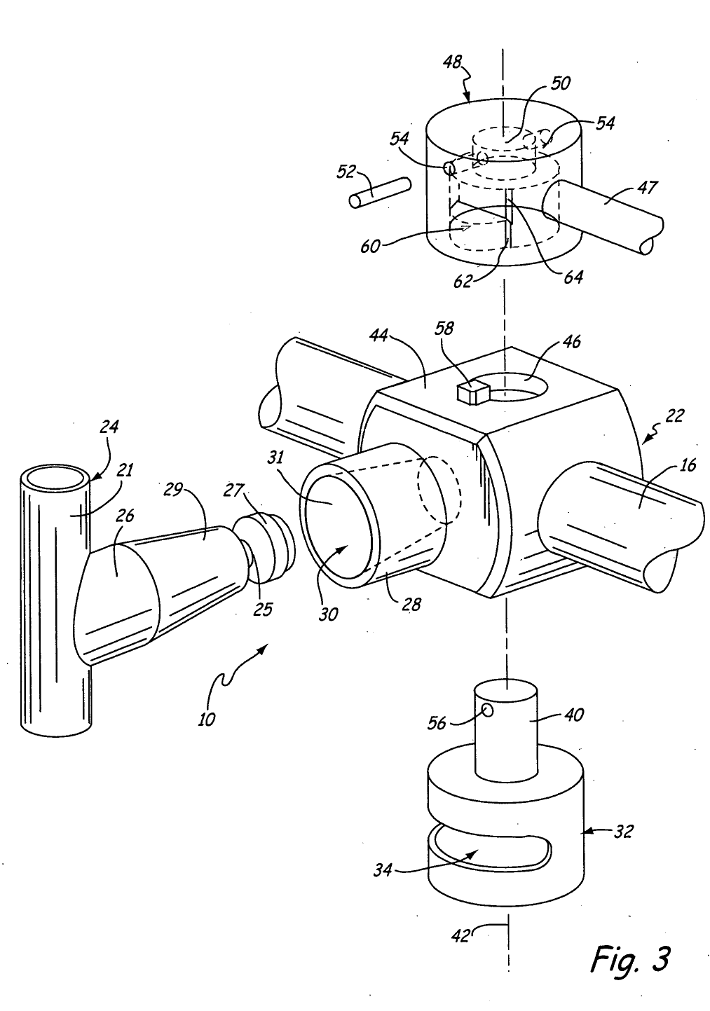

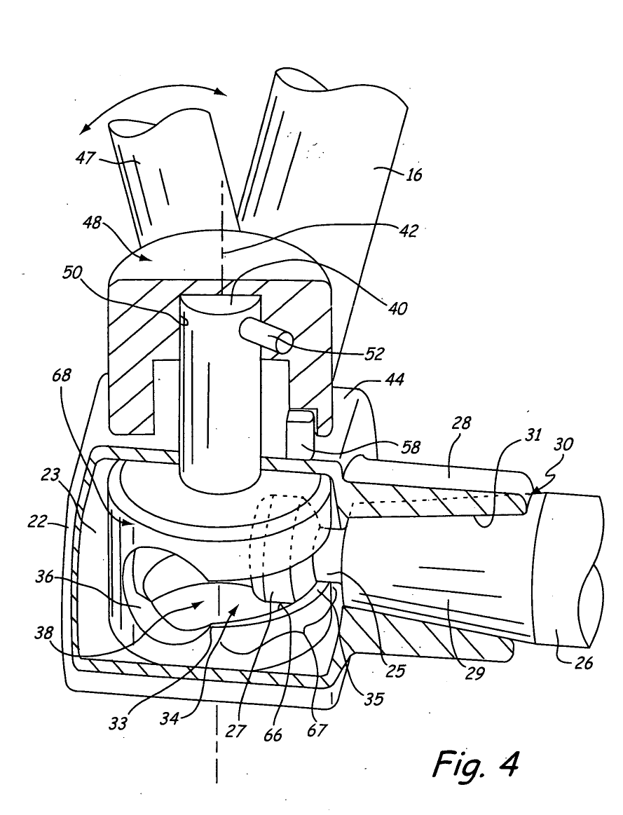

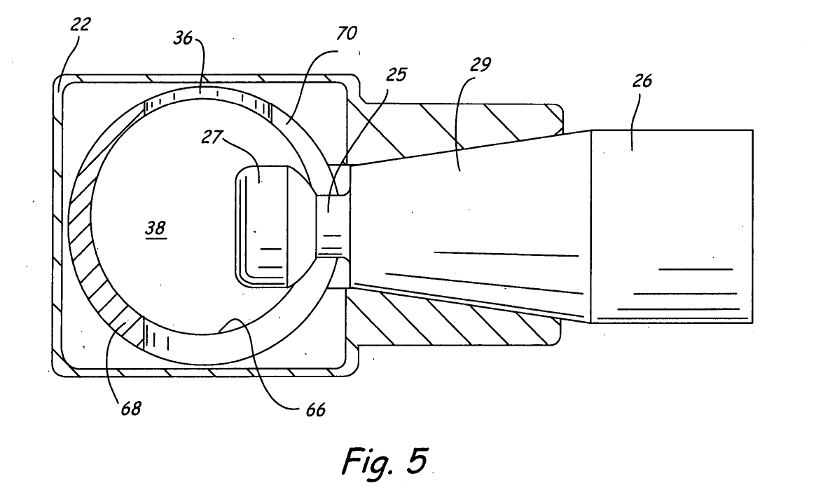

Referring to FIGS. 2-5, the surgical apparatus 24 is positioned in the selected position by positioning a shaft 26 of the surgical apparatus 24 through a through bore 30 of a cylindrical extension 28 that is attached to the housing 22. The through bore 30 intersects a cavity 23 of the housing 22 where the through bore 30 is aligned with a slot 34 within an actuating mechanism 32 positioned within the cavity 23 of the housing 22 as best illustrated in FIG. 4. When the actuating mechanism 32 is positioned in a first non-clamping position, a generally circular opening 36 at an end of the slot 34 aligns with the through bore 30 such that a head 27 of the shaft 26 passes through the generally circular opening 36 and into a void 38 of the actuating mechanism 32.

The head 27 of the shaft 26 preferably has a generally circular cross-section that is positionable within the generally circular opening 36. However, a different cross-sectionally configured head and a differently configured opening that accepts the head are within the scope of the present invention.

The actuating mechanism 32 includes a shaft 40 positioned about an axis 42 of the actuating mechanism 32 where the shaft 40 extends through a through hole 46 in an upper wall 44 attached to an end of the housing 22. A hub 48 is positioned over the shaft 40 where the hub 48 includes a generally cylindrical recess 50 that accepts the shaft 40. The hub 48 is attached to the shaft 40 by a pin 52 that is inserted into aligned through bores 54 positioned on opposing sides of the recess 50 and a bore 56 of the shaft 40.

With the shaft 40 fixedly attached to the hub 48 by the pin 52, the actuating mechanism 32 is captivated within the cavity 23 by the upper wall 44 and the hub 48. The upper wall 44 prevents the actuating mechanism 32 from exiting the cavity 23 through the top end of the housing 22 and the hub 48 engages the upper wall 44 to prevent the actuating mechanism 32 from falling out of the cavity 23 through a bottom end.

Rotation of the hub 48 and the actuating mechanism 32 about the axis 42 is limited by a stop 58 extending from the upper wall 44 and into an arcuate groove 60 of the generally cylindrical recess 50. The stop 58 limits movement of the hub 48 and the actuating mechanism 32 by engaging either a first end 62 or a second end 64 of the arcuate groove 60 which corresponds to the actuating mechanism 32 being in either a first non-clamping position or a second clamping position, respectively.

With the actuating mechanism 32 in the first non-clamping position, the generally circular opening 36 of the slot 32 is aligned with the through bore 30 of the cylindrical extension 28. The shaft 26 of a surgical apparatus 24 is positioned within the through bore 30 of the cylindrical extension 28 wherein the head 27 of the shaft 26 is positioned through the circular opening 36 of the slot 34 and within the void 38 of the actuating mechanism 32. With the shaft 26 positioned within the through bore 30 and the head 27 positioned within the void 38, an annular groove 25 proximate the head 27 is aligned with the slot 34.

As the actuating mechanism 32 is rotated from the first non-clamping position to the second clamping position, the annular groove 25 moves along the slot 34 and an inner surface 66 of a wall 68 defining a portion of the void 38 engages the head 27 and draws the head 27 into the void 38. The thickness of the wall 68 adjacent the slot 34 increases from a first end 33 adjacent the circular opening 36 to a location 35. A rotatable wedge portion 67 that when moved increases its thickness (or width) thereby generating a force on the head 27 to draw the head 27 within the void 38 while the annular groove 25 moves along the slot 34 as the actuating mechanism 32 is rotated from the first non-clamping position to the second clamping position.

When the actuating mechanism 32 is moved to the second clamping position, a frusto-conical surface 29 of the shaft 26 frictionally engages a frusto-conical surface 31 of the through bore 30. The location 35 is that position on the wedge portion 67 where the wedge portion 67 cannot be moved further due to the engagement of the surface 31 with the surface 29. The frictional engagement of the frusto-conical surfaces 29, 31 secures the surgical apparatus 24 in the selected position. Preferably, a conduit 21 is attached to the shaft 26, although any surgical apparatus having a similarly configured shaft 26 is securable within the surgical apparatus-retaining device 10. The conduit 21 represents conventional surgical apparatus such as, but not limited to, retractors, endoscopes, arthroscopes, surgical microscopes, bronchoscopes, colonoscopes, proctoscopes and the like.

In operation, the surgical apparatus-retaining device 10 is secured in a selected position proximate a surgical site 12 by clamping the support rod 16 to support apparatus 18. A handle 47 attached to the hub 48 is moved which rotates the actuating mechanism 32 into the first non-clamping position where the generally circular opening 36 of the slot 34 is aligned with the through bore 30 of the cylindrical extension 28.

The shaft 26 of the surgical apparatus 24 is positioned within the through bore 30 of the cylindrical extension 28 and the head 27 is positioned within the void 38 such that the annular groove 25 is aligned with the slot 34. With the actuating mechanism 32 in the first non-clamping position, the shaft 26 is rotatable within the through bore 30 and the surgical apparatus 24 is positioned into a selected position within a surgical site 12.

With the surgical apparatus 24 positioned into the selected position within the surgical site 12, the handle 47 is moved which rotates the hub 48 and the actuating mechanism 32 from the first non-clamping position toward the second clamping position. As the actuating mechanism 32 is rotated, the annular groove 25 moves along the slot 34 and the inner surface 66 engages the head 27 and draws the head 27 into the void 38.

With the actuating mechanism 32 in the second clamping position, the frusto-conical surface 29 of the shaft 26 frictionally engages the frusto-conical surface 31 of the through bore 30. The frictional engagement of the frusto-conical surfaces 29, 31 fixedly retains the surgical apparatus 24 in the selected position.

The surgical apparatus 24 is removable from the surgical site by moving the handle 47 such that the actuating mechanism 32 is positioned into the first non-clamping position. With the actuating mechanism 32 in the first non-clamping position, the circular opening 36 of the slot 34 is aligned with the through bore 36. Manual force is applied to the surgical apparatus 24 to remove the head 27 from the void 38 and the shaft 26 from the through bore 30.

The surgical apparatus 24 can be repositioned precisely in the same location in the surgical site 12 by retaining the surgical apparatus-retaining device 10 in the selected position proximate the surgical site 12. With the surgical apparatus-retaining device 10 in the selected position and the actuating mechanism 32 in the first position, the shaft 24 of the surgical apparatus is positioned within the through bore 30 with the head 27 positioned within the void 38 and the annular groove 25 aligned with the slot 34.

The actuating mechanism 32 is rotated into the second clamping position where the inner surface 66 draws the head 27 into the void 38 such that the frusto-conical surface 29 of the shaft 26 is forced into a frictional engagement with the frusto-conical surface 31 of the through bore 30. With the frusto-conical surfaces 29, 31 frictionally engaged, the surgical apparatus 24 is precisely relocated in the selected position within the surgical site 12.

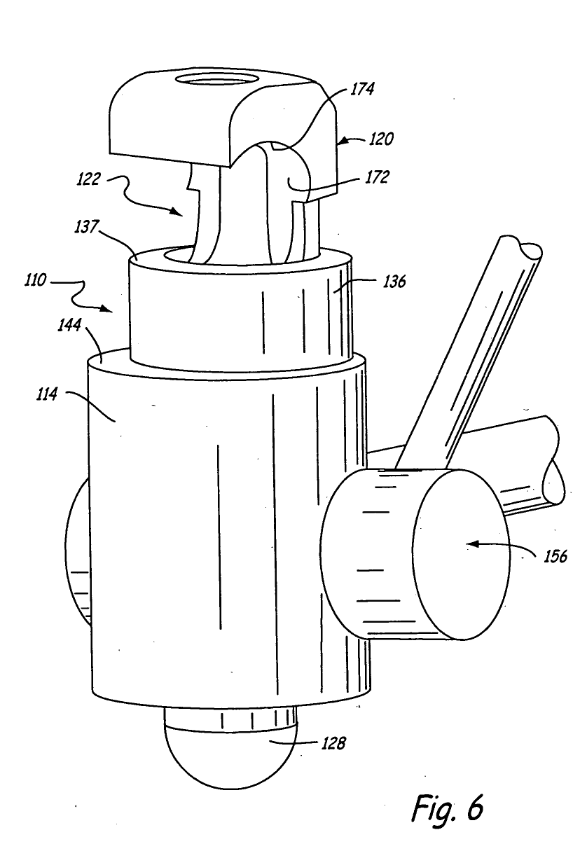

An alternative embodiment of the surgical apparatus-retaining device of the present invention is generally illustrated at 110 in FIG. 6. The surgical apparatus-retaining device 110 secures a surgical apparatus 112 in a selected location within a surgical site (not shown). A housing 114 of the surgical apparatus-retaining device 110 is fixedly attached to an end of a support rod 116 that extends over the surgical site where the support rod 116 is attached to a support apparatus with a clamp as previously illustrated in FIG. 1.

The surgical apparatus 112 is positioned within a socket 122 of a clamping member 120 extending from the housing 114 by applying manual force substantially perpendicular to a back surface 124 of the socket 122 (or within a substantially 180° range from the back surface) that overcomes a bias of a compression spring 126 positioned between the clamping member 120 and a follower 128 positioned within an internal void 115 of the housing 114. By socket is meant an opening or a cavity into which an inserted part, such as a surgical apparatus, is designed to fit and wherein the surgical apparatus can be inserted into the socket from an infinite number of directions in a 180° range starting from a substantially parallel position to the back surface of the socket to a position substantially perpendicular to the back surface and continuing to position again substantially parallel to the back surface of the socket.

The clamping member 120 is operably attached to the follower 128 with a threaded bolt 130 positioned through a through bore 132 in the clamping member 120 that threadably engages a threaded bore 134 within the follower 128. The clamping member 120 is positioned within the housing 114 through a bore 138 of a bushing 136.

The bushing 136 includes a cylindrical portion 140 positioned within an internal cavity 115 of the housing 114 through a through hole 142 of a plate 144 that is attached to the housing 114. An annular groove 146 on the cylindrical portion 140 is positioned within the internal cavity 115 and a snap ring 148 is positioned within the annular groove 146 to rotatably captivate the bushing 136 to the housing 114.

An extension 150 of the clamping member 120 is positioned within a slot 152 of the bushing 136 proximate the through bore 138. The extension 150 has a substantially complementary configuration to a surface 154 defining the slot 152 where the extension 150 engages the surface 154 to rotatably fix the clamping member 120 in relationship to the bushing 136.

With the surgical apparatus 112 with the socket 122, the compression spring 126 biases the clamping member 120 toward the bushing 136 to constrict an opening 123 of the socket 122 and retain the surgical apparatus 112 within the socket 122. With the surgical apparatus 112 retained within the socket 122, the surgical apparatus 112 is slidably positionable along an axis 113 and is rotatable along with the bushing 136 and the clamping member 120 with respect to the housing 114 such that the surgical apparatus 112 is positioned into a selected position.

The surgical apparatus 112 is fixed in the select position with a camming pin 156 positioned through through bores 117 of the housing 114 opposite each other and a through bore 127 of the follower 128. The camming pin 156 includes proximal and distal end portions 158, 160 and an intermediate portion 162 that are adjacent one to another. The proximal end portion 158 and the intermediate portion 162 are generally cylindrical in configuration. The distal end portion 160 has arcuate portions 164 separated by flat sides 166 that allow the distal end portion 160 to pass through the through bore 127 within the follower 128. The proximal and distal end portions 158, 162 are centered about a rotational axis 168 of the camming pin 156 and are captivated within the first and second through bores 117.

The captivated proximal and distal end portions 158, 162 rotatably support the intermediate portion 162 within the through bore 127 of the follower 128 where the intermediate portion 162 is eccentrically coupled between the proximal and distal end portions 158, 162. The intermediate portion 162 includes a camming surface 161 and has an axis 170 that is spaced from the rotational axis 168 the camming pin 156 by a selected distance. The distance separating the rotational axis 168 of the camming pin 156 and the axis 170 of the intermediate portion 160 generally determines the maximum distance that the camming pin 156 moves the follower 128 within the internal void 115 of the housing 114.

As the camming pin 156 is moved from a first non-clamping position where the surgical apparatus 112 is positionable within the surgical site to a second clamping position, the camming surface 161 of the intermediate portion 160 engages the through bore 127 of the follower 128 and forces the follower 128 away from the bushing 136 and the clamping member 120. As the follower 128 is forced away from the bushing 136 and the clamping member 120, a head 131 of the bolt 130 engages a shoulder 133 of the through bore 134 and forces the clamping member 120 into the bushing 136.

The force created by the bolt 130 upon the clamping member 120 creates the frictional engagement between an arcuate upper portion 174 of a clamping surface 172, the surgical apparatus 112 and a top surface 137 of the bushing 136. The surgical apparatus 112 also forces the bushing 136 into a frictional engagement between the plate 144 and the surgical apparatus 112.

To remove the surgical apparatus 112 from the surgical apparatus-retaining device 116, the camming pin 156 is positioned from the second clamping position to the first non-clamping position where the follower 128 is forced toward the bushing 136 and the clamping member 120 and wherein the head 131 of the bolt 130 disengages the shoulder 133. With the camming pin 156 in the first non-clamping position, the compression spring 126 biases the clamping member 120 toward the clamping position to slidably retain the surgical apparatus 112 within the socket 122. With the camming pin 156 in the first position, the bushing 136 is rotatable within the housing 114, the clamping member 160 is slidable within the bushing 136 and the surgical apparatus 112 is removable from the socket 122 with manual force.

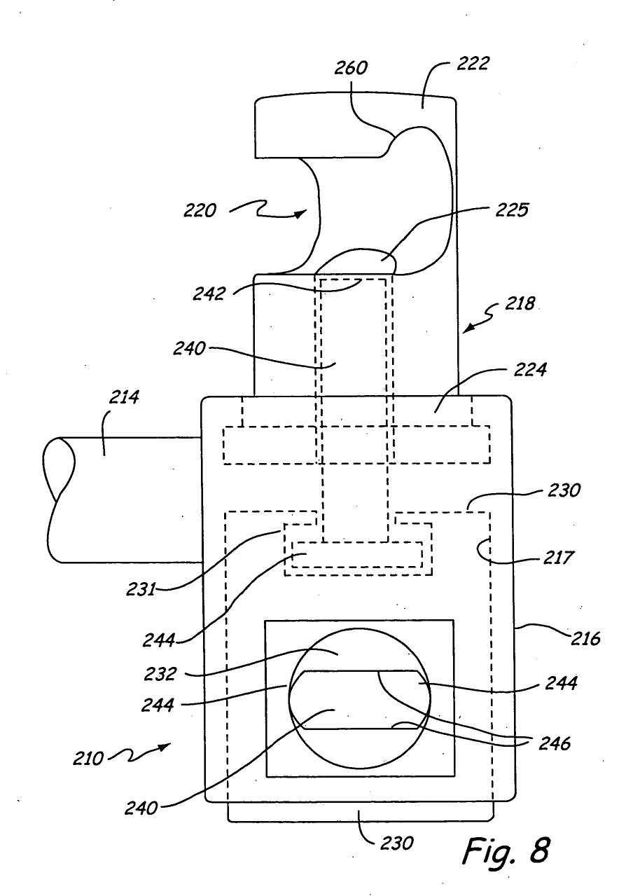

Another alternative embodiment of the surgical apparatus-retaining device of the present invention is generally illustrated in FIG. 8 at 210. The surgical apparatus-retaining device 210 secures a surgical apparatus 212 in a selected location within a surgical site. A housing 216 of the surgical apparatus-retaining device 210 is fixedly attached to an end of a support rod 214 that extends over the surgical site where the support rod 214 is fixedly attached to a support apparatus (not shown) with a clamp (not shown).

The surgical apparatus 212 is positioned within a socket 220 within a distal portion 222 of a clamping member 218. A proximal cylindrical portion 224 of a clamping member is positioned within an internal cavity 217 of the housing 216 between a follower 230 and a lip 226 of the housing 216 where the cylindrical portion 224 is rotatable within internal cavity 217.

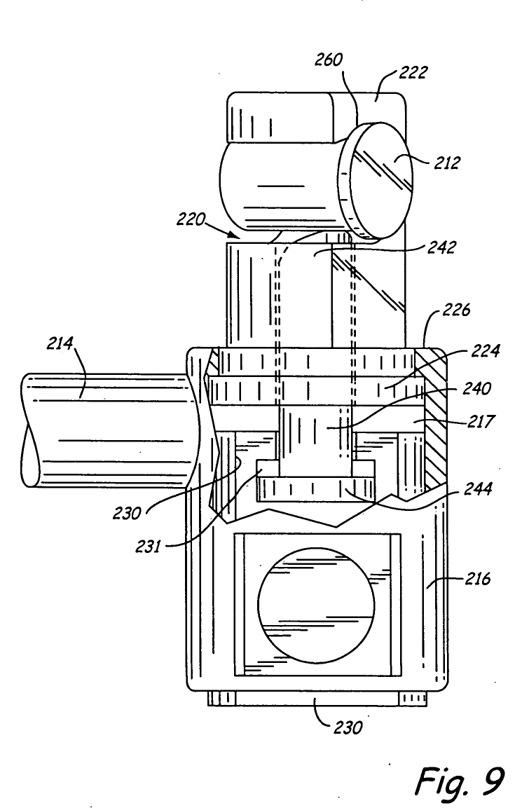

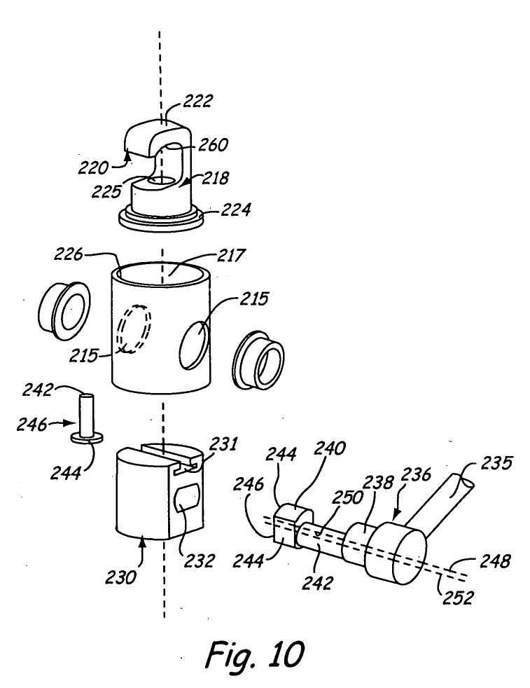

Referring to FIGS. 8-10, the follower 230 includes a through bore 232 that is aligned with through bores 215 opposite each other within the housing 216. A camming pin 236 is positioned through the bores 215 of the housing 216 and the through bore 232 of the follower 230.

A bore 225 axially located within the cylindrical portion 224 intersects the socket 220. A clamping pin 240 is disposed within the bore 225 where an end 242 of the clamping pin 240 is disposed proximate the socket 220 and a head 244 of the clamping pin 240 is positioned within a “T” shaped channel 231 within the follower 230. The “T” shaped channel 231 engages the head 244 and retains the clamping pin 240 to the follower 230.

The surgical apparatus 212 is fixed in a selected position by positioning the camming pin 236 from a first non-clamping position where the surgical apparatus 212 is movable within the socket 220 to a second clamping position where the surgical apparatus 212 is secured within the socket 220 in a selected position. The camming pin 236 includes proximal and distal end portions 238, 240 and an intermediate portion 242 that are adjacent one to another. The proximal end portion 238 and the intermediate portion 242 are generally cylindrical in shape. The distal end portion 240 has arcuate portions 244 separated by flat sides 246 that allow the distal end portion 240 to pass through the through bore 232 within the follower 230. The proximal and distal end portions 238, 240 are centered about a rotational axis 248 of the camming pin 236 and are captivated within the first and second through bores 215.

The captivated proximal and distal end portions 238, 240 rotatably support the intermediate portion 242 within the through bore 232 of the follower 230 where the intermediate portion 242 is eccentrically coupled between the proximal and distal end portions 238, 240. The intermediate portion 242 includes a camming surface 250 and has an axis 252 that is spaced from the rotational axis 240 of the camming pin 236 by a selected distance. The distance separating the rotational axis 248 of the camming pin 236 and the axis 252 of the intermediate portion 242 generally determines the maximum distance that the camming pin 236 moves the follower 230 within the housing 216.

As the camming pin 236 is rotated from a non-clamping to a clamping position, the follower 230 is forced into contact with the cylindrical portion 224 wherein the cylindrical portion 224 is frictionally engaged between the follower 230 and the lip 226. The follower 230 engages the head 244 of the shaft 240, positioned within the “T” shaped channel 231, and forces the end 242 into the socket 220 such that the surgical apparatus 212 is frictionally engaged between an arcuate upper surface 260 defining a portion of the socket 220 and the end 242 of the shaft 240.

In operation, the surgical apparatus-retaining device 210 is secured in a selected position about a surgical site by clamping the support rod 216 to the support apparatus (not shown). The camming pin 236 is positioned into the first non-clamping position wherein the end 242 of the shaft 240 is positioned away from the socket 220. The surgical apparatus 212 is positioned within the socket 220 and the clamping member 218 is rotated within the housing 216 such that the surgical apparatus 212 is in the selected position.

A handle 235 which is attached to the camming pin 236 is moved such that the camming pin 236 is rotated from the first non-clamping position to the second clamping position. As the camming pin 236 is rotated into the second clamping position, the follower 230 is forced into the cylindrical portion 224 and frictionally engages the cylindrical portion 224 between the lip 226 and the follower 230. The frictional engagement of the cylindrical portion 224 between the follower 230 and the lip 226 fixes the position of the clamping member 218 with respect to the housing 216.

As the follower 230 is moved into contact with the cylindrical portion 224, the follower 230 engages the head 244 of the shaft 240 and forces the end 242 into the socket 220. The surgical apparatus 212 is frictionally engaged between the arcuate upper surface 260 and the end 242 of the shaft 240.

The surgical apparatus 212 is removed from the socket 220 by rotating the camming pin 236 from the second clamping position to the first non-clamping position where the end 242 of the shaft 240 is removed from the socket 220. With the end 242 removed from the socket 240, the surgical apparatus 212 is removed from the socket 220 with manual force.

The surgical apparatus 212 is repositionable in the selected position by positioning the surgical apparatus 212 in the socket 240 and rotating the camming pin 236 from the first non-clamping to the second clamping position. Provided the surgical apparatus-retaining device 210 remains stationary proximate the surgical site, the surgical apparatus 212 is repositionable in the selected position.

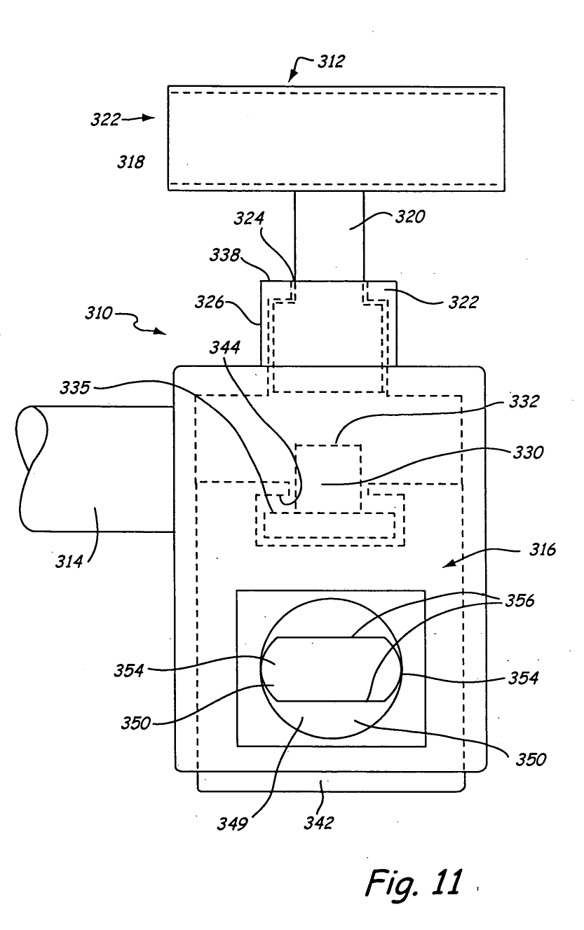

Another alternative embodiment of the clamping device of the present invention is generally illustrated in FIG. 11 at 310. The surgical apparatus-retaining device 310 secures a surgical apparatus 312 in a selected location within a surgical site. A housing 316 of the surgical apparatus-retaining device is fixedly attached to an end of a support rod 314 that extends over a surgical site where the support rod 314 is attached to a support apparatus (not shown) with a clamp (not shown).

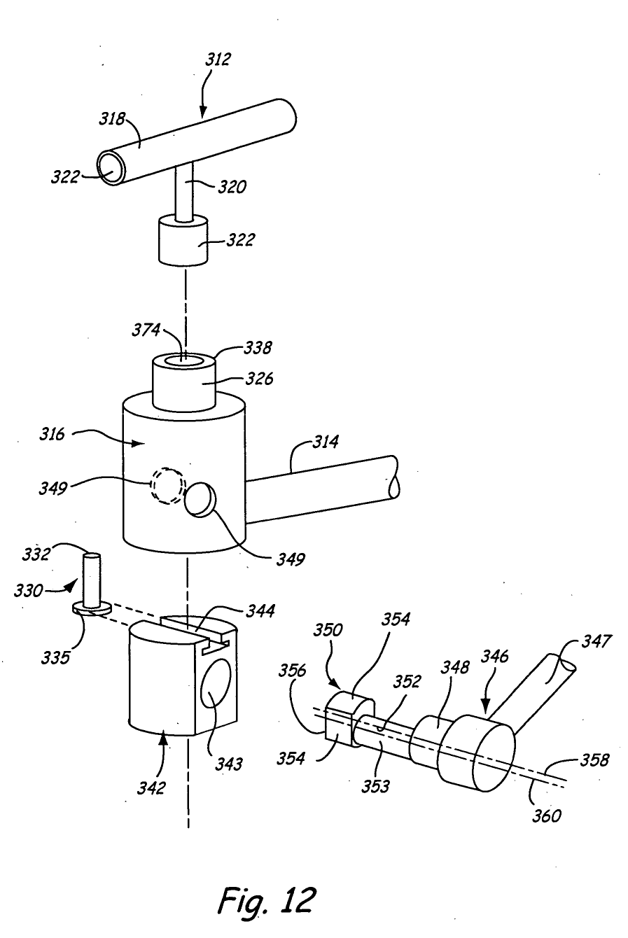

Referring to FIGS. 11 and 12, the surgical apparatus 312 includes a conduit 318. A through bore 322 of the conduit 318 provides access to the surgical site and retains another surgical apparatus (not shown) within the surgical site.

The conduit 318 is attached to a cylindrical portion 322 of the surgical apparatus 316 with a shaft 320 positioned through a constricted opening 324 of a cylindrical extension 326 attached to the housing 316. The cylindrical portion 322 is rotatably positionable within the cylindrical extension 326.

The surgical retaining apparatus 312 is secured in a selected position by forcing an end 332 of a clamping pin 360 operably attached to a follower 342 into contact with the cylindrical portion 322 and creating a frictional engagement between a lip 336 constricting the opening 324 to the cylindrical extension 326, the cylindrical portion 322 and the end 332 of the clamping pin 330.

A head 335 of the clamping pin 330 is positioned within a “T” shaped channel 344 of the follower 342. As the follower 342 is moved into a clamping position with a camming pin 346, a surface 343 defining the “T” shaped channel 344 engages the head 335 of the pin 330 and forces the end 332 of the clamping pin 330 into the cylindrical portion 324. The cylindrical portion 324 is fixed in a selected position by a frictional engagement between the shoulder 338 and the end 332 of the clamping pin 330.

The camming pin 346 is positioned through the bores 349 of the housing 316 and a through bore 343 of the follower 342. The camming pin 346 includes proximal and distal end portions 348, 350 and an intermediate portion 352 that are adjacent one to another. The proximal end portion 348 and the intermediate portion 352 are generally cylindrical in shape. The distal end portion 350 has arcuate portions 354 separated by flat sides 356 that allow the distal end 350 portion to pass through the through bore 343 within the follower 342. The proximal and distal end portions 348, 350 are centered about a rotational axis 358 and are captivated within the first and second through bores 349.

The captivated proximal and distal end portions 348, 350 rotatably support the intermediate portion 352 within the through bore 343 of the follower 342 where the intermediate portion 352 is eccentrically coupled between the proximal and distal end portions 348, 350. The intermediate portion 352 includes a camming surface 353 and has an axis 360 that is spaced from the rotational axis 358 the camming pin 346 by a selected distance. The distance separating the rotational axis 358 of the camming pin 346 and the axis 360 of the intermediate portion 352 generally determines the maximum distance that the camming pin 346 moves the follower 342 within the housing 316.

With the camming pin 346 a first non-clamping position the cylindrical portion 324 is rotatable within the cylindrical extension 326. With the camming pin 346 in a second clamping position the cylindrical portion 324 is secured between the lip 338 and the end 332 of the clamping pin 330.

In operation, the surgical apparatus-retaining device 310 is secured in a selected position within a surgical site by clamping the support rod 314 to the support apparatus (not shown). A handle 347 which is attached to the camming pin 346 is moved which rotates the camming pin 346 into the first position where the surgical apparatus 312 is rotatable with respect to the housing. With the camming pin 346 in the first position, the surgical apparatus 312 is rotated into a selected position.

With the surgical apparatus 312 in the selected position, the camming pin 346 is rotated into the second clamping position. In the second clamping position the cylindrical portion 322 of the surgical apparatus 312 is frictionally engaged between the lip 338 and the end 332 of the clamping pin 330.

Although the present invention has been described with reference to preferred embodiments, workers skilled in the art will recognize that changes may be made in form and detail without departing from the spirit and scope of the invention.

1. A device for positioning a surgical apparatus in a selected position within a surgical site, the device comprising:
a rod for positioning over the surgical site;
a housing attached to the rod the housing having an internal cavity;
a clamping mechanism attached to the housing and wherein the clamping mechanism includes a bore for engaging the surgical apparatus; and
a force providing mechanism movably positioned within the internal cavity of the housing wherein the force providing mechanism moves to secure the surgical apparatus to the clamping mechanism in the selected position within the surgical site.

2. The device of claim 1 wherein the force providing mechanism comprises a rotatable wedge wherein the rotatable wedge moves to secure the surgical apparatus to the clamping mechanism in the selected position within the surgical site.

3. The device of claim 1 wherein the force providing mechanism comprises a camming surface wherein the camming surface moves to secure the surgical apparatus to the clamping mechanism in the selected position within the surgical site.

4. The device of claim 1 and further comprising a clamping pin operably connected to the force providing mechanism wherein the force providing mechanism moves an end of the pin to engage the surgical apparatus within the socket to frictionally secure the surgical apparatus in the selected position within the surgical site.

5. The device of claim 1 wherein the bore comprises a frusto-conical surface wherein the surgical apparatus frictionally engages the frusto-conical surface to secure the surgical apparatus within the surgical site.

6. The device of claim 1 and further comprising a handle attached to the force providing mechanism.

7. A clamp for securing a surgical apparatus in a selected position within a surgical site, the clamp comprising:
a housing having an internal cavity;
a clamping mechanism comprising a socket wherein the clamping mechanism operably attaches to the housing and wherein the surgical apparatus is positioned within the socket; and
an actuating mechanism positioned within the internal cavity and in communication with the clamping mechanism and wherein the actuating mechanism moves to secure the surgical apparatus to the clamping mechanism.

8. The clamp of claim 7 wherein the actuating mechanism comprises a rotatable wedge and wherein the rotatable wedge moves to secure the surgical apparatus to the clamping mechanism.

9. The clamp of claim 7 wherein the actuating mechanism comprises a camming surface and wherein as the camming surface moves to secure the surgical apparatus to the clamping mechanism.

10. The clamp of claim 7 wherein the socket comprises a frusto-conical surface that engages the surgical apparatus and secures the surgical apparatus in a select position as the actuating mechanism is moved.

11. The clamp of claim 7 wherein the socket constricts to frictionally engage the surgical apparatus as the actuating mechanism is moved.

12. The clamp of claim 7 and further comprising a clamping pin operably attached to the actuating mechanism and wherein the actuating mechanism moves the pin to engage the surgical apparatus within the socket to secure the surgical apparatus in the selected position within the surgical site.

13. The clamp of claim 7 and further comprising a handle attached to the actuating mechanism.

14. The clamp of claim 7 and further comprising a rod attached to the housing for securing the clamp in a selected position.

15. A device for precisely repositioning a surgical apparatus in a selected position within a surgical site, the device comprising:
a support rod positionable above the surgical site;
a clamping mechanism supported by the rod and wherein the clamping mechanism comprises a socket for engaging the surgical apparatus; and
a force providing mechanism in communication with the clamping mechanism wherein the force providing mechanism is positionable between a first position wherein the surgical apparatus is removable from the socket and a second position wherein the surgical apparatus is secured within the socket of the clamping mechanism and wherein with the surgical rod stationarily positioned within the surgical site, the surgical apparatus is removable from the surgical site and repositionable in the selected position within the surgical site.

16. The device of claim 15, wherein the force providing mechanism comprises a rotatable wedge when the rotatable wedge moves between the first position wherein the surgical apparatus is removable from the socket and the second position wherein the surgical apparatus is secured within the socket.

17. The surgical device of claim 15, wherein the force providing mechanism comprises a camming surface wherein the camming surface moves between the first position wherein the surgical apparatus is removable from the socket and the second position wherein the surgical apparatus is secured within the socket.

18. The surgical device of claim 15, wherein the socket comprises a frusto-conical surface that engages the surgical apparatus to frictionally secure the surgical apparatus within the surgical site.

19. The clamp of claim 15 and further comprising a clamping pin operably attached to the force providing mechanism and wherein the force providing mechanism moves the pin to engage the surgical apparatus within the socket to secure the surgical apparatus in the selected position within the surgical site.

20. A clamp for clamping a surgical apparatus having an end portion, the clamp comprising:
a receiving unit configured for engaging the end portion of the surgical apparatus; and
a clamping mechanism engageable with the end portion for drawing the end portion within the receiving unit such that the end portion is held in a clamped position.

21. The clamp of claim 20 and wherein the receiving unit frictionally engages the end portion to hold the end portion in the clamped position.

22. The clamp of claim 20 and wherein the receiving unit comprises a through bore having a first frusto-conical surface.

23. The clamp of claim 22 and wherin the end portion comprises a second frusto-conical surface that frictionally engages the first frusto-conical surface of the through bore to hold the end portion in the clamped position.

24. The clamp of claim 20 and wherein the clamping mechanism comprises a rotatable wedge wherein the rotatable wedge moves to hold the end portion in the clamped position.

25. The clamp of claim 20 and wherein the clamping mechanism comprises a camming surface wherein the camming surface moves to hold the end portion in the clamped position.
